(19) United States
(12) Patent Application Publication
(10) Pub. No.: US 2022/0273707 A1
(43) Pub. Date: Sep. 1, 2022

(54) COMPOSITIONS AND METHODS FOR TREATING CANINE PARVOVIRUS INFECTION

(71) Applicant: MoxxiTech, LLC, Allen, TX (US)

(72) Inventors: Krystin DEASON, Lucas, TX (US); Wayne R. DEASON, Floresville, TX (US)

(73) Assignee: MoxxiTech, LLC, Allen, TX (US)

(21) Appl. No.: 17/679,698

(22) Filed: Feb. 24, 2022

Related U.S. Application Data
(60) Provisional application No. 63/153,230, filed on Feb. 24, 2021.

(51) Int. Cl. A61K 33/26; A61K 47/26; A61P 31/20

(52) U.S. Cl. CPC A61K 33/26; A61K 47/26; A61P 31/20

(57) ABSTRACT

Provided herein are therapies that decrease the symptoms associated with Canine parvovirus infection, decrease time to recovery, and decrease the time infected dogs spend hospitalized in isolation.

### REFERENCE TO RELATED APPLICATIONS

The present application claims the priority benefit of U.S. provisional application No. 63/153,230, filed Feb. 24, 2021, the entire contents of which is incorporated herein by reference.

### BACKGROUND

### 1. Field

The present invention relates generally to the field of veterinary medicine. More particularly, it concerns compositions and methods for treatment of Canine parvovirus.

### 2. Description of Related Art

Canine parvovirus is a particularly deadly disease among young puppies, about 80% fatal, causing gastrointestinal tract damage and dehydration as well as a cardiac syndrome in very young pups. It is spread by contact with an infected dog's feces. Symptoms include lethargy, severe diarrhea, fever, vomiting, loss of appetite, and dehydration. Current treatments for Canine parvovirus infections are non-specific, mainly involving supportive care and the prevention of secondary infections, and typically require lengthy hospitalizations in isolated rooms to prevent the spread of the highly contagious virus. New therapies are needed to treat dogs with Canine parvovirus infections.

### SUMMARY

In one embodiment, provided herein are pharmaceutical compositions comprising an iron containing compound, an excipient, and optionally glucose. In some aspects, the iron containing compound is a ferric iron containing compound. In some aspects, the ferric iron containing compound is ferric citrate. In some aspects, the glucose is D-glucose. In some aspects, the pharmaceutical compositions are formulated for oral administration.

In some aspects, the pharmaceutical compositions further comprise a salt solution. In some aspects, the salt solution comprises one or more of sodium chloride, potassium chloride, potassium phosphate monobasic, sodium phosphate dibasic, magnesium sulfate, calcium chloride, and sodium bicarbonate. In some aspects, the pharmaceutical composition comprises ferric citrate, sodium chloride, potassium chloride, potassium phosphate monobasic, sodium phosphate dibasic, magnesium sulfate, calcium chloride, sodium bicarbonate, and D-glucose.

In one embodiment, provided herein are methods of treating a disease or disorder in a subject in need thereof, the method comprising administering a therapeutically effective amount of the pharmaceutical composition of any one of the present embodiments to the subject. In some aspects, the subject has a viral infection with a virus that uses the iron transport pathway. In some aspects, the subject has a Parvoviridae infection. In some aspects, the subject is a canine that has a parvovirus infection. In some aspects, the canine is a young puppy and/or weighs less than 5 lbs. In some aspects, the methods shorten the course of the disease. In some aspects, the methods ameliorate many of the symptoms of Canine parvovirus infection including vomiting, diarrhea, lethargy, and loss of appetite. In some aspects, the canine further has a hookworm infection. In some aspects, the administering is performed orally. In some aspects, the administering is performed twice daily. In some aspects, the subject is a feline that has a Parvoviridae infection. In some aspects, the subject is a human that has an Arenaviridae infection. In some aspects, the subject has a bacterial infection with a pathogenic bacterium that uses siderophores to sequester iron. In some aspects, the subject has gastroenteritis or anemia.

Other objects, features, and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows that iron availability improves cell survival in the presence of CPV infection.

FIG. 2 shows that in the absence of CPV viral infection, added ferric citrate does not alter cell viability.

FIG. 3 shows that treatment of dogs with increasing concentrations of ferric citrate results in increased survival from natural infections with CPV.

FIG. 4 shows that there is no significant difference between the median weight of puppies treated with each concentration of ferric citrate.

### DETAILED DESCRIPTION

Provided herein are solutions comprising an iron containing compound, such as, for example, ferric citrate, in a salt solution with glucose. Also provided herein are methods of treating Canine parvovirus with the solutions. In vitro, the solution protects from Canine parvovirus induced cell death. The solution, when provided orally to dogs naturally infected with Canine parvovirus, ameliorates many of the symptoms of Canine parvovirus infection including vomiting, diarrhea, lethargy, and loss of appetite, and decreases the course of the disease, with full recovery beginning as quickly as 12 hours after the initial treatment.

### I. CANINE PARVOVIRUS

As discussed further below, current treatments for Canine parvovirus infections are non-specific, mainly involving supportive care and the prevention of secondary infections, and typically require lengthy hospitalizations in isolated rooms to prevent the spread of the highly contagious virus. Provided herein are specific therapies that decrease the symptoms associated with Canine parvovirus infection, decrease time to recovery, and decrease the time infected dogs spend hospitalized in isolation.

Canine parvovirus (also referred to as CPV, CPV2, or parvo) is a contagious DNA virus of the Parvoviridae family that mainly affects dogs. However, CPV may infect other mammals including foxes, wolves, cats, and skunks. CPV is highly contagious and is spread from dog to dog by direct or indirect contact with their feces. Vaccines can prevent this infection, but mortality can reach 91% in untreated cases. Treatment often involves veterinary hospitalization.

There are two types of CPV called canine minute virus (CPV1) and CPV2. CPV2 causes the most serious disease and affects domesticated dogs and wild canids. With severe disease, dogs can die within 48 to 72 hours without treatment by fluids. In the more common, less severe form, mortality is about 10 percent. Factors such as age, breed, a stressful environment, concurrent infections with bacteria, parasites, and canine coronavirus increase a dog's risk of severe infection.

Dogs that develop the disease show signs of the illness within three to seven days. The signs may include lethargy, vomiting, fever, and diarrhea (usually bloody). Generally, the first sign of CPV is lethargy. Secondary signs are loss of weight and appetite or diarrhea followed by vomiting. Diarrhea and vomiting result in dehydration that upsets the electrolyte balance, which may critically affect the dog. Secondary infections occur as a result of the weakened immune system. Because the normal intestinal lining is also compromised, blood and protein leak into the intestines, leading to anemia and loss of protein, and endotoxins escape into the bloodstream, causing endotoxemia.

Diagnosis is made through detection of CPV in the feces by either an ELISA or a hemagglutination test, or by electron microscopy. PCR has become available to diagnose CPV and can be used later in the disease when potentially less virus is being shed in the feces that may not be detectable by ELISA.

The survival rate depends on how quickly CPV is diagnosed, the age of the dog, and how aggressive the treatment is. There is no approved treatment, and the current standard of care is supportive care, involving extensive hospitalization, due to severe dehydration and potential damage to the intestines and bone marrow.

### II. THERAPEUTIC COMPOSITIONS

Provided herein are therapeutic compositions comprising an iron containing compound, such as, for example, ferric citrate, in a salt solution, optionally with glucose.

In some aspects, the iron containing compound is a ferric iron containing compound, such as, for example, ferric citrate. Ferric citrate, or iron(III) citrate, describes any of several complexes formed upon binding any of the several conjugate bases derived from citric acid with ferric ions. These complexes contain two or more Fe(III) centers. In some aspects, the iron containing compound is ferrous ascorbate, ferrous carbonate, ferrous citrate, ferrous fumarate, ferrous gluconate, ferrous lactate, ferrous succinate, ferrous sulfate, ferrous glycine sulfate, ferric ammonium citrate, ferric chloride, ferric oxide, ferric phosphate, ferric polymaltose, ferric protein succinylate, ferric pyrophosphate, ferric sodium pyrophosphate, or ferric sulfate.

In some aspects, the therapeutic composition further comprises a salt solution comprises one or more of sodium chloride (NaCl), potassium chloride (KCl), potassium phosphate monobasic (KH₂PO₄), sodium phosphate dibasic (Na₂HPO₄), magnesium sulfate (MgSO₄), calcium chloride (CaCl₂), and sodium bicarbonate (NaHCO₃).

In some aspects, the therapeutic composition further comprises glucose, such as, for example, D-glucose.

In some aspects, the therapeutic composition comprises ferric citrate (C₆H₅FeO₇), sodium chloride (NaCl), potassium chloride (KCl), potassium phosphate monobasic (KH₂PO₄), sodium phosphate dibasic, anhydrous (Na₂HPO₄), magnesium sulfate, anhydrous (MgSO₄), calcium chloride, anhydrous (CaCl₂.2H₂O), sodium bicarbonate (NaHCO₃), and D-glucose.

In some aspects, the therapeutic composition comprises 2-50 g/L (e.g., 2, 3, 4, 5, 6, 7, 8, 9, 10, 12, 14, 16, 18, 20, 25, 30, 35, 40, 45, or 50 g/L, or any value derivable therein, such as, for example, 15 g/L) ferric citrate (C₆H₅FeO₇), 0-0.5 g/L (e.g., 0.01, 0.1, 0.2, 0.3, 0.4, or 0.5 g/L, or any value derivable therein, such as, for example, 0.185 g/L) sodium chloride (NaCl), 0-0.5 g/L (e.g., 0.01, 0.1, 0.2, 0.3, 0.4, or 0.5 g/L, or any value derivable therein, such as, for example, 0.4 g/L) potassium chloride (KCl), 0-0.2 g/L (e.g., 0.01, 0.02, 0.05, 0.1, or 0.2 g/L, or any value derivable therein, such as, for example, 0.06 g/L) potassium phosphate monobasic (KH₂PO₄), 0-0.2 g/L (e.g., 0.01, 0.02, 0.05, 0.1, or 0.2 g/L, or any value derivable therein, such as, for example, 0.048 g/L) sodium phosphate dibasic, anhydrous (Na₂HPO₄), 0-0.2 g/L (e.g., 0.01, 0.02, 0.05, 0.1, or 0.2 g/L, or any value derivable therein, such as, for example, 0.098 g/L) magnesium sulfate, anhydrous (MgSO₄), 0-0.5 g/L (e.g., 0.01, 0.1, 0.2, 0.3, 0.4, or 0.5 g/L, or any value derivable therein, such as, for example, 0.185 g/L) calcium chloride, anhydrous (CaCl₂.2H₂O), 0-0.5 g/L (e.g., 0.01, 0.1, 0.2, 0.3, 0.4, or 0.5 g/L, or any value derivable therein, such as, for example, 0.35 g/L) sodium bicarbonate (NaHCO₃), and 0-5 g/L (e.g., 0.1, 0.5, 1, 2.5, or 5 g/L, or any value derivable therein, such as, for example, 1 g/L) D-glucose.

In some aspects, the therapeutic composition is formulated for oral administration. The therapeutic compound may be prepared as a liquid concentrate, which may be packaged in individual (i.e., single-use) unit dose or in multi-use dose formats. Concentrated liquid formulations may comprise the therapeutic agent and a solvent (e.g., an organic or aqueous solvent). A liquid composition for oral administration may be obtainable by mixing the concentrated liquid formulation with an aqueous medium.

In another aspect, for administration to a patient in need of such treatment, therapeutic compositions (also referred to as a pharmaceutical preparations, pharmaceutical compositions, pharmaceutical formulations, pharmaceutical products, medicinal products, medicines, medications, or medicaments) comprise a therapeutically effective amount of an iron containing compound disclosed herein formulated with one or more excipients and/or drug carriers appropriate to the indicated route of administration. In some embodiments, the iron containing compounds disclosed herein are formulated in a manner amenable for the treatment of human and/or veterinary patients. In some embodiments, formulation comprises admixing or combining one or more of the iron containing compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted, encapsulated, or in liquid form. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

In one embodiment, the therapeutic composition is prepared by dissolving ferric citrate (C₆H₅FeO₇) in a solution comprising sodium chloride (NaCl), potassium chloride (KCl), potassium phosphate monobasic (KH₂PO₄), sodium phosphate dibasic, anhydrous (Na₂HPO₄), magnesium sulfate, anhydrous (MgSO₄), calcium chloride, anhydrous (CaCl₂.2H₂O), sodium bicarbonate (NaHCO₃), and D-glucose.

In some aspects, the liquid oral therapeutic compositions comprise at least one buffering agent. Examples of buffering agents include, but are not limited to, sodium bicarbonate, potassium bicarbonate, magnesium hydroxide, magnesium lactate, magnesium gluconate, other magnesium salts, aluminum hydroxide, aluminum hydroxide/sodium bicarbonate coprecipitate, a mixture of an amino acid and a buffer, a mixture of aluminum glycinate and a buffer, a mixture of an acid salt of an amino acid and a buffer, and a mixture of an alkali salt of an amino acid and a buffer. Additional buffering agents include sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, sodium acetate, potassium metaphosphate, magnesium oxide, magnesium hydroxide, magnesium carbonate, magnesium silicate, calcium acetate, calcium glycerophosphate, calcium chloride, calcium hydroxide, calcium lactate, calcium carbonate, calcium bicarbonate, and other calcium salts.

In some aspects, the liquid oral therapeutic compositions comprise at least one pharmaceutically acceptable carrier. The pharmaceutically acceptable carrier of the liquid oral therapeutic compositions may comprise a bicarbonate salt of Group IA metal as buffering agent, and can be prepared by mixing the bicarbonate salt of the Group IA metal, preferably sodium bicarbonate, with water. The concentration of the bicarbonate salt of the Group IA metal in the composition generally ranges from approximately 5.0 percent to approximately 60.0 percent.

Additionally, various additives can be incorporated into the therapeutic composition to enhance its stability, sterility, and isotonicity. Antimicrobial preservatives, antioxidants, chelating agents, and additional buffers can be added. Various antibacterial and antifungal agents such as, for example, parabens, chlorobutanol, phenol, sorbic acid, and the like can enhance prevention of the action of microorganisms.

In some cases, it may be desirable to include isotonic agents, for example, sugars, sodium chloride, and the like. Additionally, thickening agents, such as methyl cellulose, may be desirable.

In some aspects, the therapeutic composition can alternatively be formulated as a powder, tablet, suspension tablet, chewable tablet, capsule, two-part tablet or capsule, effervescent powder, effervescent tablet, pellets, and granules.

Pharmaceutical formulations may be administered by a variety of methods, e.g., orally or by injection (e.g. subcutaneous, intravenous, and intraperitoneal). Depending on the route of administration, the iron containing compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions.

The iron containing compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The iron containing compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The iron containing compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

### III. METHODS OF TREATMENT

Provided herein are methods of treating certain viral infections, bacterial infections, and/or anemias with solutions comprising an iron containing compound, such as, for example, ferric citrate, in a salt solution with glucose. In a preferred embodiment, the methods treat Canine parvovirus. In other embodiments, the methods treat other viruses that also utilize the iron transport pathway (by binding to the transferrin receptor, for example), including other members of the Parvoviridae family, such as the Feline panleukopenia virus, and members of the Arenaviridae family, which cause acute viral hemorrhagic fever in humans.

In some aspects, the methods treat conditions of certain pathogenic bacterial infections, as many pathogenic bacteria utilize siderophores to sequester iron from their host, which may result in the host becoming iron deficient either locally (to the infection) or systemically. Localized iron availability is critical for macrophage function (such as through the production of reactive oxygen species) and CD4+ T cell activation and proliferation, for example. In the case of bacterial infections, the treatment may be applied orally, as with the canine parvovirus infection therapy, to treat gut-specific or systemic infections, or may be applied locally, in combination with antibiotic therapy.

In some aspects, the methods treat gastroenteritis of undefined origin (Canine parvovirus negative). In another aspects, the methods treat dogs that have become anemic due to hookworm infections and are therefore at a greater risk for severe complications from canine parvovirus infections because hookworms cause significant intestinal distress and anemia as they attach directly to the intestinal wall where they draw blood from their host (dog).

In a preferred embodiment, the therapeutic compositions provided herein are used to treat Canine parvovirus infections, including very young/small puppies (<5 lbs) that otherwise quickly succumb to the parvovirus infection despite hospitalization. In this preferred embodiment, the therapeutic composition is provided is an easy to dispense solution for oral administration. In some aspects, the method increases the chance of survival of the patient.

Such treatment may also be in combination with another therapeutic regime, such as supportive care. Supportive care may consist of crystalloid IV fluids and/or colloids (e.g., Hetastarch), antinausea injections (antiemetics) such as maropitant, metoclopramide, dolasetron, ondansetron and prochlorperazine, and broad-spectrum antibiotic injections such as cefazolin/enrofloxacin, ampicillin/enrofloxacin, metronidazole, timentin, or enrofloxacin. IV fluids may be administered and antinausea and antibiotic injections may be given subcutaneously, intramuscularly, or intravenously. The fluids may be a mix of a sterile, balanced electrolyte solution, with an appropriate amount of B-complex vitamins, dextrose, and potassium chloride. Analgesic medications may be used to counteract the intestinal discomfort caused by frequent bouts of diarrhea.

Once the dog can keep fluids down, the IV fluids can be gradually discontinued, and very bland food slowly introduced. Oral antibiotics may be administered for a number of days depending on the white blood cell count and the patient's ability to fight off secondary infection.

Single or multiple doses of the therapeutic compositions are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

In various aspects, a preferred dosage amount is 0.1-10 mg/kg, 0.22-10 mg/kg, 0.1-5.5 mg/kg, or 0.22-5.5 mg/kg ferric citrate. The dosage may be 0.1, 0.2, 0.3, 0.4, 0.5, 0.6, 0.7, 0.8, 0.9, 1.0, 1.5, 2, 2.5, 3, 3.5, 4, 4.5, 5, 5.5, 6, 6.5, 7, 7.5, 8, 8.5, 9, 9.5, or 10 mg/kg ferric citrate, or any value derivable therein, such as, for example 0.22, 1.1, or 2.2 mg/kg. The dosage may be administered twice daily for 1, 2, 3, 4, 5, or more days.

The therapeutic compositions may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the therapeutic compositions may be taken orally and that the timing of which is or is not dependent upon food intake.

Precise amounts of the therapeutic composition administered depend on the judgment of the practitioner and are specific to each patient. Various factors affecting the dose include the physical and clinical state of the patient, the route of administration, the intended goal of treatment, and the potency, stability and toxicity of the particular therapeutic formulation.

The actual dosage amount of a therapeutic composition of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

### IV. DEFINITIONS

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “therapeutically effective amount,” or “pharmaceutically effective amount” when used in the context of treating a patient or subject with an active ingredient means that amount of the active ingredient which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio. Moreover, for animal administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

A “therapeutic composition” (also referred to as a pharmaceutical, pharmaceutical preparation, pharmaceutical composition, pharmaceutical drug, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) and optionally contains one or more inactive ingredients, which are also referred to as excipients.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

A patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

“Canine”, as used herein, includes what is commonly called the dog, but includes other members of the family Canidae.

As used herein, the term “patient” or “subject” refers to a living mammalian organism, such as a human, monkey, cow, sheep, goat, dog, cat, fox, wolf, skunk, mouse, rat, guinea pig, or transgenic species thereof. In certain embodiments, the patient or subject is a canine.

“Oral” administration, as used herein, refers to the introduction of a substance, such as a therapeutic composition, into a subject's body through or by way of the mouth and involves swallowing or transport through the oral mucosa (e.g., sublingual or buccal absorption) or both. Intratracheal is also a means of oral administration.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—In Vitro Studies

Methods: Canine fibroblast cells were cultured in Dulbecco's modified Eagle's medium (DMEM) supplemented with 5% fetal bovine serum, 2 mM L-glutamine, 0.1 mM MEM non-essential amino acids, 1 mM sodium pyruvate, 0.05 mM 2-mercaptoethanol, 10 mM HEPES, 100 units/mL penicillin, and 100 mcg/mL streptomycin, at 38° C. with 5% CO₂. Canine parvovirus type 2 (CPV2) was isolated from a fecal sample collected from a naturally infected dog. The virus was propagated on canine fibroblast cells and a titer of 10^{13.37} tissue culture infectious doses (TCID50)/mL was used for viability assays. For viability assays, cells were routinely passaged and seeded in 96 well plates at 5×10⁴ cells/mL in 200 μL complete DMEM media and incubated overnight at 38° C. with 5% CO₂. The media was then removed and cells were infected with CPV2 for 1 hour at 38° C. with 5% CO₂. After 1-hour, ferric citrate (1 μM or 10 μM), deferoxamine (1 μM), or fresh media was added to the wells in triplicate and cells were incubated for 72 hours then analyzed for cell viability using trypan blue exclusion. A minimum of 3 independent experiments were performed for each assay.

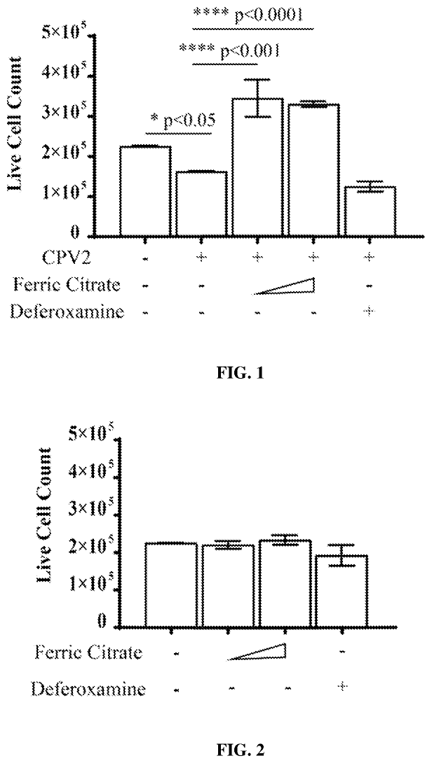

To determine the role of iron availability during CPV infection, ferric citrate or deferoxamine was added to cell cultures inoculated with CPV. 72 hours post treatment, cells were assayed for viability. Cells infected with CPV alone showed a 28% decrease in survival, compared to untreated cells (FIG. 1). Increased availability of iron, through the addition of 1 μM or 10 μM ferric citrate resulted in a significant increase in cell viability after infection with CPV while decreased availability of iron, through the addition of 1 μM deferoxamine, led to a 45% decrease in survival, compared to the untreated cells, or 23% decrease in survival compared to cells infected with CPV. This suggests that the availability of iron during CPV infection is critical for cell survival. Data shown are Mean±SEM, pooled from three independent experiments.

To determine whether additional ferric citrate affects cell survival in the absence of CPV infection, cells were treated with 1 μM or 10 μM ferric citrate or 1 μM deferoxamine for 72 hours, then assayed for cell survival. In all cases there was no significant difference between the viability of untreated and treated cells (FIG. 2). Data shown are Mean±SEM, pooled from three independent experiments.

### Example 2—In Vivo Studies

The solution, when provided orally to dogs naturally infected with Canine parvovirus ameliorates many of the symptoms of Canine parvovirus infection including vomiting, diarrhea, lethargy, and loss of appetite, and decreases the course of the disease, with full recovery beginning as quickly as 12 hours post initial treatment.

Methods: Thirteen puppies with no history of vaccination against CPV developed CPV infections through natural routes of infection and were treated following standard veterinary protocol supplemented with 0.22, 1.1, or 2.2 mg/kg ferric citrate solution orally, twice daily for up to five days, and monitored for 14 days.

Puppies presenting with symptoms of CPV infection, including vomiting, diarrhea, hematochezia, lethargy, and depression, were tested for CPV infection using IDEXX Laboratories SNAP® Parvo Test per manufacturer's protocols. Once confirmed to be CPV positive, puppies began treatment following standard veterinary protocols for CPV infection, including intravenous or subcutaneous fluids, antiemetics, and injectable antibiotics, supplemented with 0.22, 1.1, or 2.2 mg/kg ferric citrate solution.

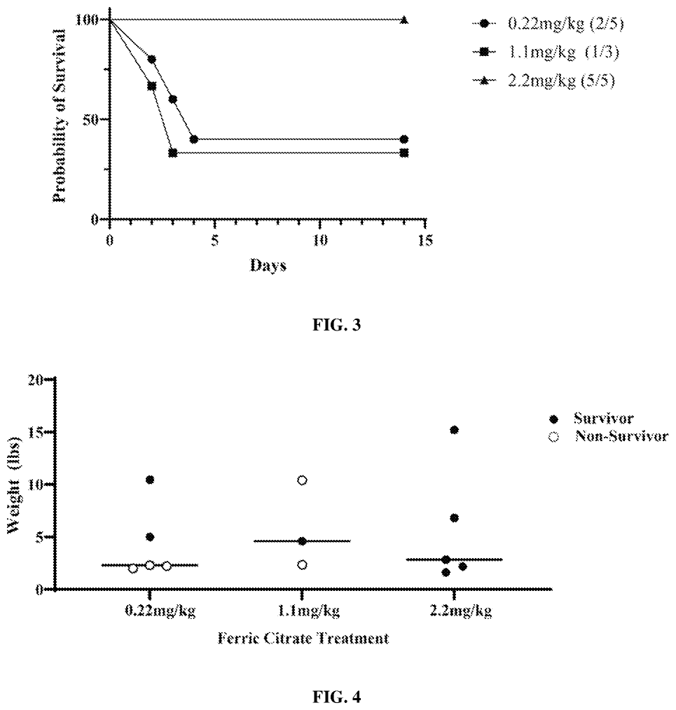

The puppies supplemented with the highest concentration of ferric citrate had 100% survival rate (5/5), while the survival rate of the lower concentrations was 40% (⅖) and 33.33% (⅓), respectively (FIG. 3).

Young puppies with low body weights (<5 lbs) are at high risk of mortality when infected with CPV. While the lowest concentration of ferric citrate tested, 0.22 mg/kg, was not able to prevent mortality of the smallest puppies (<5 lbs), the highest concentration tested, 2.2 mg/kg, showed 100% survival of all puppies, the smallest weighing only 1.6 lbs at the beginning of treatment (FIG. 4).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A pharmaceutical composition comprising an iron containing compound, an excipient, and optionally glucose.

2. The pharmaceutical composition of claim 1, wherein the iron containing compound is a ferric iron containing compound.

3. The pharmaceutical composition of claim 2, wherein the ferric iron containing compound is ferric citrate.

4. The pharmaceutical composition of claim 1, wherein the glucose is D-glucose.

5. The pharmaceutical composition of claim 1, further comprising a salt solution.

6. The pharmaceutical composition of claim 5, wherein the salt solution comprises one or more of sodium chloride, potassium chloride, potassium phosphate monobasic, sodium phosphate dibasic, magnesium sulfate, calcium chloride, and sodium bicarbonate.

7. The pharmaceutical composition of claim 1, wherein the pharmaceutical composition comprises ferric citrate, sodium chloride, potassium chloride, potassium phosphate monobasic, sodium phosphate dibasic, magnesium sulfate, calcium chloride, sodium bicarbonate, and D-glucose.

8. The pharmaceutical composition of claim 1, wherein the composition is formulated for oral administration.

9. A method of treating a disease or disorder in a subject in need thereof, the method comprising administering a therapeutically effective amount of the pharmaceutical composition of claim 1 to the subject.

10. The method of claim 9, wherein the subject has a viral infection with a virus that uses the iron transport pathway.

11. The method of claim 9, wherein the subject has a Parvoviridae infection.

12. The method of claim 9, wherein the subject is a canine that has a parvovirus infection.

13. The method of claim 12, wherein the canine is a young puppy and/or weighs less than 5 lbs.

14. The method of claim 12, wherein the method shortens the course of the disease.

15. The method of claim 12, wherein the method ameliorates many of the symptoms of Canine parvovirus infection including vomiting, diarrhea, lethargy, and loss of appetite.

16. The method of claim 12, wherein the canine further has a hookworm infection.

17. The method of claim 9, wherein the administering is performed orally.

18. The method of claim 9, wherein the subject is a feline that has a Parvoviridae infection.

19. The method of claim 9, wherein the subject is a human that has an Arenaviridae infection.

20. The method of claim 9, wherein the subject has a bacterial infection with a pathogenic bacterium that uses siderophores to sequester iron.

21. The method of claim 9, wherein the subject has gastroenteritis or anemia.
